(19) United States
(12) Patent Application Publication
(10) Pub. No.: US 2011/0189626 A1
(43) Pub. Date: Aug. 4, 2011

(54) TEETH WHITENING SYSTEM, APPARATUS, AND RELATED METHOD

(75) Inventor: Martin Angelo Sanzari, Elmwood Park, NJ (US)

(73) Assignee: Engineered Cosmetic Solutions, LLC, South Riding, VA (US)

(21) Appl. No.: 12/696,372

(22) Filed: Jan. 29, 2010

(51) Int. Cl. A61C 19/06; A61N 5/06

(52) U.S. Cl. 433/29; 433/25; 433/215

(57) ABSTRACT

A teeth whitening system includes a head unit adapted to be worn on a user's head, a light housing supported by the head unit proximate to the user's mouth, the light housing including a light source, and a mouth piece including a tray portion adapted to surround the user's teeth. At least one of the light housing or the mouth piece includes a receptacle, and the other of the light housing or the mouth piece includes a plug portion adapted to engage in the receptacle. The present application also provides an apparatus for whitening teeth, a mouth piece for use in whitening teeth, and a method of whitening teeth.

### BACKGROUND

(1) Technical Field

The present invention relates generally to the field of teeth whitening, and more specifically, to a system, apparatus, and method for hands free, portable, and/or in-home teeth whitening.

(2) Related Art

Over the years, the desire for white teeth has grown among all age groups. As the populace has become more health conscious, there has been an increased desire to look younger and healthier. Generally it is preferred to have white teeth rather than to have dark, stained, or discolored teeth. A higher standard of living has brought the ability to have cosmetic enhancements to the forefront.

The natural shape of teeth and their color among the populace is very diverse. The color of the teeth usually ranges from whites, grays, browns, and yellows. Many factors can affect the color of teeth including nutrition, medications, and the ingestion of blueberries, coffee, tea, and other foods with the tendency to stain the teeth. The teeth can also become strained over time as a result of smoking, chewing tobacco, or taking Tetracycline or other medications.

The ability to whiten teeth with peroxides is well known, and many materials have been devised to whiten an individuals teeth using chemicals. Chromogen molecules are generally responsible for the discoloration of the teeth. This discoloration can be removed through the use of an oxidizing agent, such as hydrogen peroxide or carbamide peroxide, in the form of liquids, pastes, and gels. Oxidation renders the chromogen molecules colorless resulting in whiter and brighter teeth.

The most commonly accepted chemicals used in teeth whitening today are peroxides which are highly reactive and considered physiologically safe as compared to other oxidizing agents. Some peroxides of choice for teeth whitening are: hydrogen peroxide, carbamine peroxide, sodium perborate, and sodium percarbonate. When these peroxides are in appropriate contact with the teeth, they will usually oxidize both the internal and external organic stains, rendering the teeth whiter. In contrast, organic stains resulting from some medications are usually not affected by peroxides.

Since the use of peroxides has been generally accepted for use in teeth whitening, a multitude of methods for applying peroxides have been devised. One of the common ways to apply a bleaching paste is the use of a dental tray which holds the bleach in contact with the teeth, but prevents the bleach from flowing away from the teeth. The dental tray may be an arched shaped container made to conform to the shape of the teeth and mouth. It can be used retain the paste around the teeth, thus ensuring that the paste remains in contact with the teeth. This also reduces the time that a patient must spend in a dental chair, and it removes the use of uncomfortable cheek retractors. However, dental trays are usually opaque to visible light and are of limited use for procedures which require activation by visible light irradiation.

Manufacturers have developed systems that use oxidizing compositions containing up to 35% hydrogen peroxide, which are only available from a dentist in the office. A 35% hydrogen peroxide composition is a strong oxidizer and can be hazardous to the patient if not administered properly. Systems using this composition are typically applied directly to the surface of the teeth in the form of a thick paste. This type of system usually requires approximately one hour per application to whiten the teeth. These whitening systems are typically limited to use in the dentist's office, and are expensive to the patient.

In view of the foregoing, there is a need for improved methods and apparatuses for whitening teeth that are capable of whiting the teeth rapidly and inexpensively, and that can be used by the consumer at home without harming the gum tissue, or causing tissue irritation.

### SUMMARY

According to an illustrative embodiment, a teeth whitening system comprises a head unit adapted to be worn on a user's head; a light housing supported by the head unit proximate to the user's mouth, the light housing including a light source; and a mouth piece including a tray portion adapted to surround the user's teeth, wherein at least one of the light housing or the mouth piece includes a receptacle, and the other of the light housing or the mouth piece includes a plug portion adapted to engage in the receptacle.

According to another illustrative embodiment, a teeth whitening apparatus comprises a head unit adapted to be worn on a user's head; a light housing supported by the head unit proximate to the user's mouth, the light housing including a light source, the light housing adapted to couple with a mouth piece; and a switch located on the light housing, wherein the switch is adapted to activate the light source upon coupling of the light housing with the mouth piece.

According to another illustrative embodiment, a mouth piece for use in whitening teeth comprises a tray portion including an inner wall and an outer wall adapted to surround a user's teeth; and at least one of a plug portion or a receptacle extending from the tray portion to a position located outside the user's mouth, wherein at least one of the plug portion or the receptacle includes a surface adapted to engage a switch on a light housing to activate a light source.

According to another illustrative embodiment, a method of whitening teeth comprises inserting a mouth piece into a user's mouth; placing a head unit on a user's head, the head unit supporting a light housing; engaging the mouth piece with the light housing; and channeling light from a light source located in the light housing to the user's teeth through the mouth piece.

Further aspects, objectives, and advantages, as well as the structure and function of exemplary embodiments, will become apparent from a consideration of the description, drawings, and examples.

### BRIEF DESCRIPTION OF THE DRAWINGS

The foregoing and other features and advantages of the invention will be apparent from the following drawings wherein like reference numbers generally indicate identical, functionally similar, and/or structurally similar elements.

FIG. 1 is a perspective view of an illustrative embodiment of a teeth whitening system according to the present invention, shown positioned on a user's head;

FIG. 2 is a side view of the teeth whitening system of FIG. 1;

FIG. 3 is a perspective view of an illustrative head unit of the teeth whitening system;

FIG. 4 is an exploded view of an illustrative light housing of the head unit shown in FIG. 3;

FIG. 5 is a perspective view of a portion of the head unit of FIG. 3;

FIG. 6 is an exploded view of an illustrative power supply compartment of the head unit of FIG. 3;

FIG. 7 is a perspective view of an illustrative mouth piece of the teeth whitening system of FIG. 1;

FIG. 8 is a top view of the mouth piece of FIG. 7;

FIG. 9 is a side view of the mouth piece of FIG. 7;

FIG. 10 is a top view of the mouth piece of FIG. 7 engaged with the light housing of FIG. 4;

FIG. 11 is a front view of the light housing of FIG. 4;

FIG. 12 is a top view of an alternative embodiment of the mouth piece of FIG. 7; and

FIG. 13 is a top view of an another alternative embodiment of the mouth piece of FIG. 7, shown in conjunction with an alternative embodiment of the light housing of FIG. 4.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without departing from the spirit and scope of the invention.

Illustrative embodiments of the invention may provide a light source for teeth whitening that can be worn as a headset by the consumer in a manner to uniformly illuminate the user's teeth, for example, through a light guiding mouth piece. This may produce a uniform increase in temperature of a teeth whitening gel located in the mouth piece and/or of the surfaces of the teeth. The increase in temperature of the whitening gel may speed up the oxidation process, resulting in a complete teeth whitening process that takes less time than prior systems, for example, in approximately twenty to forty minutes.

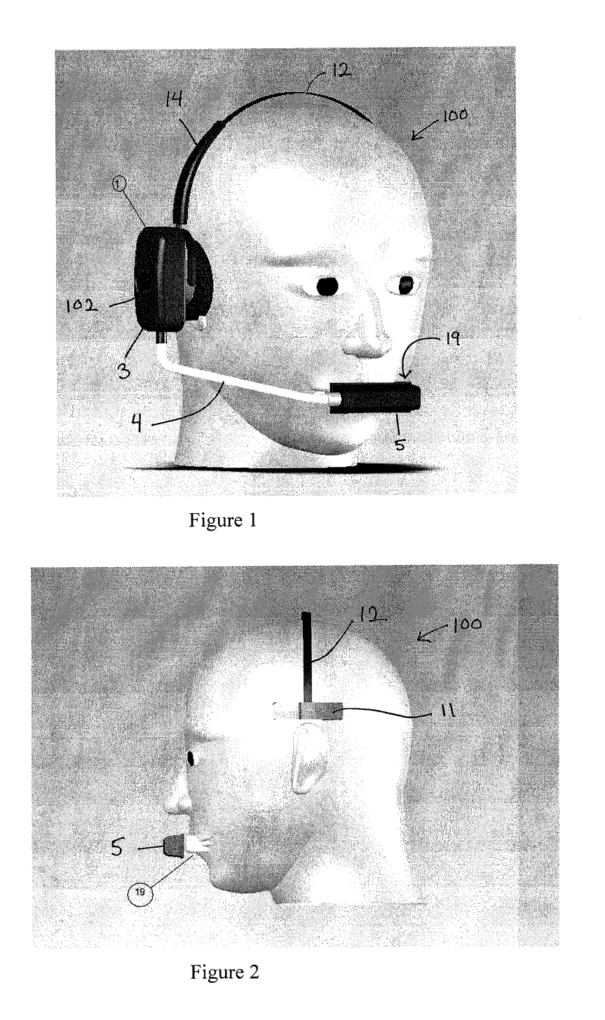

Referring to FIGS. 1 and 2, an illustrative teeth whitening system 100 according to the present invention is shown positioned on a user's head. Illustrative embodiments of the system 100 may accelerate the whitening of teeth, for example, by using a whitening agent in combination with light energy. In addition, illustrative embodiments of the system 100 may be used at home by the consumer, thereby providing more cost effective whitening than conventional teeth whitening regimes, which are typically confined to the dentist's office. Furthermore, as shown in FIG. 1, due to the configuration of illustrative embodiments, the system 100 may be portable, and/or may be supported on the head hands-free.

Still referring to FIGS. 1 and 2, the system 100 can generally include a head unit 1 that can be supported hands-free on the head, and a light guiding mouth piece 19 for placement in the user's mouth. The mouth piece 19 may contain a teeth whitening agent (not shown), such as a gel, to be discussed in more detail below. The head unit 1 can be used to activate the whitening agent. The head unit 1 can additionally or alternatively be used to irradiate the surface of the teeth with light to have an additional whitening effect by breaking down chromogens in the teeth.

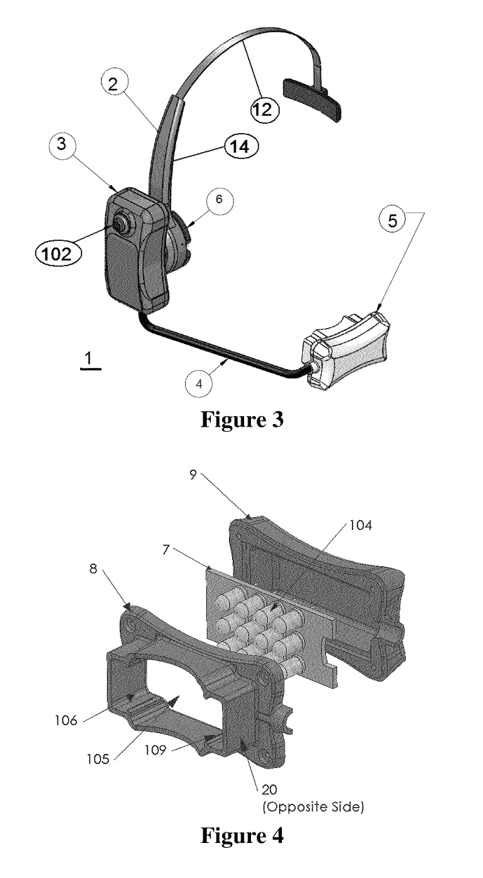

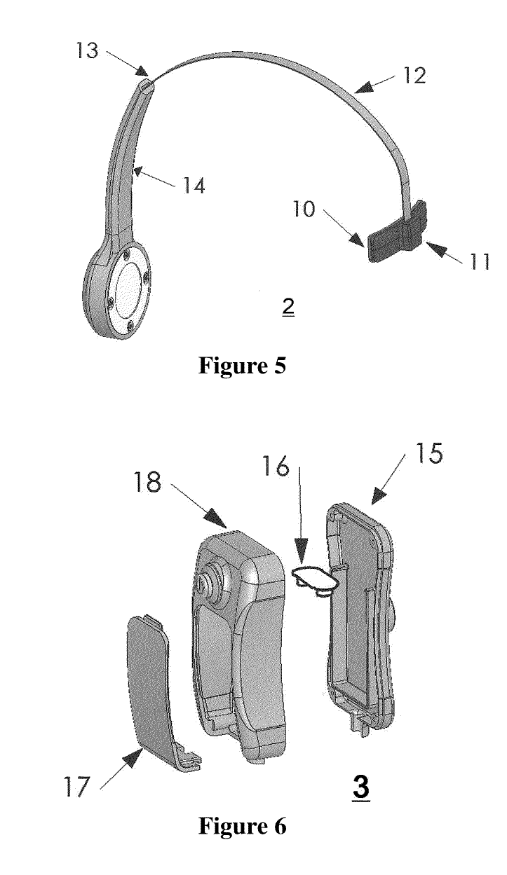

As shown in FIG. 3, the head unit 1 may include a power supply compartment 3, a head piece 2, an extension member 4, adjuster foam 6, and light housing 5. As shown in FIG. 5, the head piece 2 can comprise a base member 14, an adjustable head band 12 (e.g., of tempered steel) that extends from the base member 14, and a side head support 11. A sealing joint 13 can be located between the base member 14 and the head band 12, for example, to prevent hair from getting caught between the base member 14 and the head band 12 when adjusting the head unit 1 to fit comfortably on the user's head. A foam 10 can be provided on the side head support 11, for example, to provide comfort to the user.

Referring to FIGS. 3 and 5, the base member 14 and the head band 12 can serve as the primary support to stabilize the power supply compartment 3 on the user's head. The side head support 11 can act as support point for the head unit 1, creating a hinging point to balance the head unit 1. The side head support foam 10 can provide a comfortable support interface for the user. The head unit 1 can also include a second foam 6, shown in FIG. 3, which may act as a soft contact point and main support of the weight of the power supply compartment 3.

Referring to FIG. 1, the extension member 4 may comprise a flexible tube for positioning the light housing 5 to fit comfortably in the front of the user's mouth, where it can be coupled to mouth piece 19. The extension member 4 may be hollow so as to contain electrical wires that power the light source, such as LEDs, contained in the light housing 5. According to an illustrative embodiment, the extension member 4 may be both flexible and rigid so that it can be freely adjusted to any position in front of the mouth, yet provide sufficient rigidity to stay in position after the final adjustment is complete. Referring to FIG. 1, the head unit 1 as a whole can be weight balanced front-to-back such that its center of mass location results in the head unit 1 being comfortably balanced on the head.

Referring to FIG. 6, an illustrative embodiment of the power supply compartment 3 is shown. The compartment 3 can be used to house a power source, such as a battery. For example, a rechargeable battery such as a NiMH, NiCD, or LiON battery may be used. Alternatively, non-rechargeable batteries can be used. Further, other power sources known in the art may also be used.

As shown in FIG. 6, the power supply compartment 3 can comprise an easily removable cover 17 for easy access to the power source, such as a battery (not shown). The compartment 3 can further comprise a rear cover housing 15 and a front cover housing 18. A battery harness, such as a 9-Volt battery strap 16, can be located in the compartment 3 and can connect to the top of a battery. According to an illustrative embodiment, a 9-volt battery may provide the high voltage needed to adequately power the light source in the light housing 5, for example, where the light source comprises LEDs. A manual on/off switch 102 (shown in FIG. 3) can be located on the compartment 3 to turn the light source on and off.

Referring to FIG. 4, an illustrative embodiment of the light housing 5 is shown in exploded view. The light housing 5 can contain a light source, such as light bulbs, lasers, LEDs, or other lights known in the art. For ease of discussion, the light source will be discussed hereinafter in connection with LEDs 104.

Still referring to FIG. 4, an illustrative embodiment of the light housing 5 can contain a rear cover 9, a front cover 8, and a LED circuit board 7 located between the covers 9, 8. The front cover 8 can define a receptacle 105 that mates with the mouth piece 19, as will be described in more detail below. As will also be discussed in more detail below, one or more on/off switches 20 for activation by the mouth piece 19 may be located in or on the light housing 5.

The circuit board 7 can contain the LEDs 104. The circuit board can further include any resistors or other electronic components that may be necessary to turn the LEDs 104 on and off. In addition, the circuit board 7 can include a circuit that limits the lower voltage level at which the light source will operate. According to an illustrative embodiment, the circuit can shut down the LEDs 104 when the power from the power source decreases to a point where the light output falls below a predetermined level, for example, a reduction of approximately 20%. This can ensure that the LEDs 104 are not operating at an intensity level less than 20% of their maximum output level. A self regulating timer (not shown) can be provided to shut down the light source a predetermined time after start-up, for example, upon completion of a pre-set recommending bleaching time. As shown in the illustrative embodiment of FIG. 4, LEDs 104 can be arranged in three rows of four LEDs, however other configurations are possible. This illustrative configuration may provide a uniform power distribution of light across the front of the user's teeth.

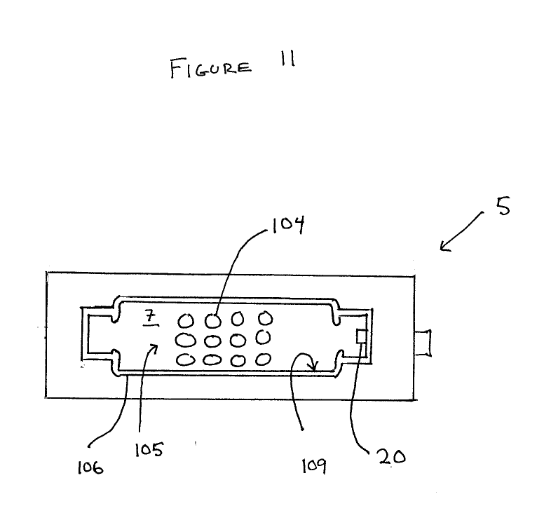

Still referring to FIG. 4, the light housing 5 can include a shroud 106 that projects around the LEDs 104 and forms part of the receptacle 105. A reflective coating 109, such as aluminum or other metallic-type reflective materials, can be located on the inner surfaces of the shroud 106 that surround the LEDs 104. This reflective coating 109 can maximize the amount of light from the LEDs 104 that passes into the mouth piece 19, and consequently, reaches the teeth and gel. Additionally or alternatively, the shroud 106 can minimize the amount of light from the LEDs 104 that shines outside the user's mouth, thereby protecting the user's eyes from light. For example, as shown in FIG. 10, when the mouth piece 19 is engaged with the light housing 5, the shroud can cover substantially all of the mouth piece 19 that extends outside of the user's mouth.

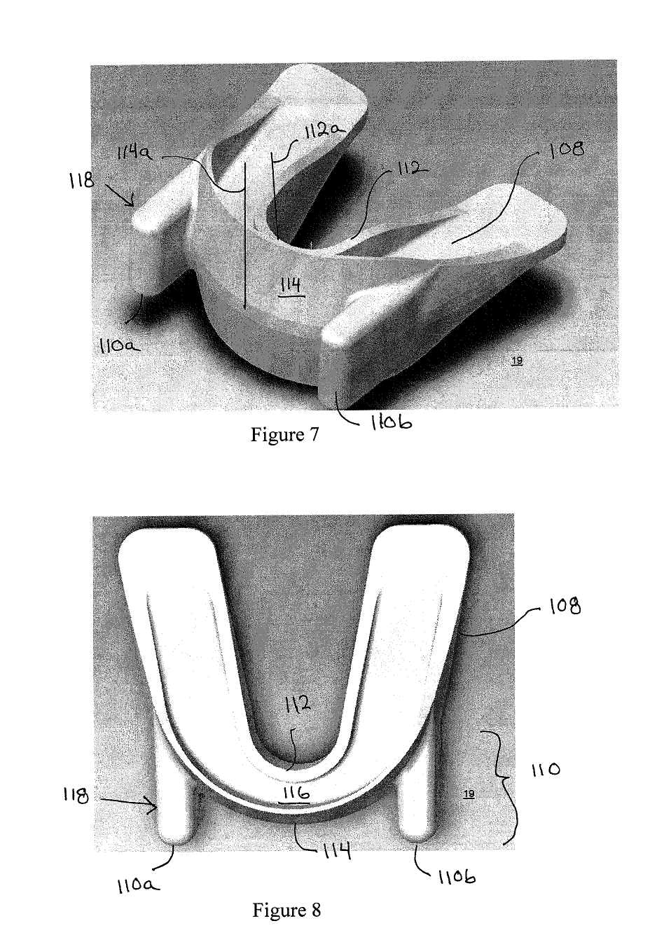

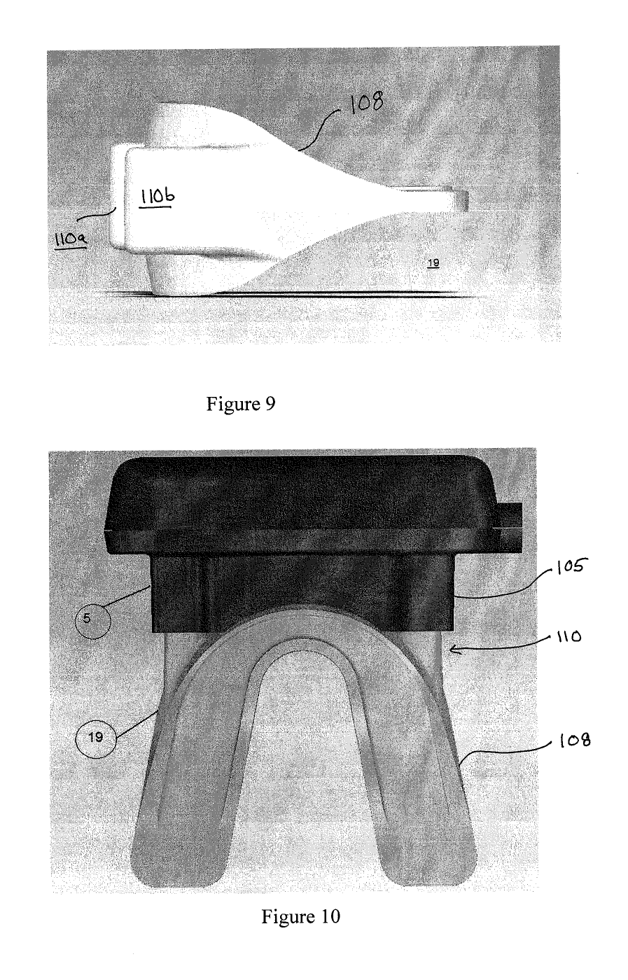

Referring to FIGS. 7-9, an illustrative embodiment of the mouth piece 19 is shown. The mouth piece 19 can be formed of a material that is transparent or translucent to light in the visible and infrared regions of the electromagnetic spectrum. This may permit light from the light housing 5 to transmit light freely through the mouth piece 19 to the teeth and any whitening agent located in the mouth piece 19. The light can act as a catalyst to the whitening agent. According to an illustrative embodiment, the mouth piece 19 can be made from urethane, silicone, or other plastics, thermoplastics, or polymers known in the art.

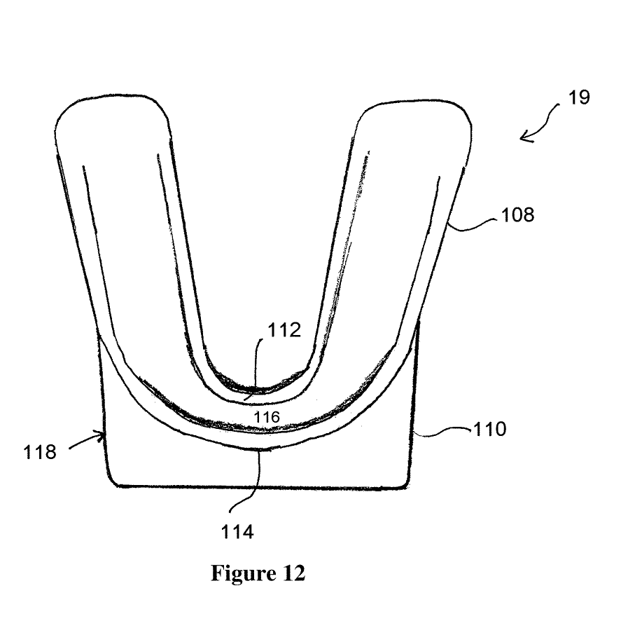

The mouth piece 19 can include a tray portion 108 that is shaped to fit within the user's mouth and surround the user's teeth. The mouth piece 19 can also include a plug portion 110 (see FIGS. 8 and 12) that extends from the tray portion 108 and is adapted to engage with the light housing 5. Referring to FIG. 8, the tray portion 108 can include an inner wall 112 and an outer wall 114 that together define a reservoir 116 for the whitening gel. FIG. 8 shows the inner wall 112, outer wall 114, and reservoir 116 located on the top side of the mouth piece 19 (e.g., for attachment to the user's upper teeth), but the inner and outer walls 112, 114 can similarly extend from the bottom side of the mouth piece 19 to define a reservoir 116 on the bottom side (e.g., for attachment to the user's lower teeth).

The inner and outer walls 112, 114 can be angled towards one another as they extend away from the reservoir 116 (e.g., out of the paper in FIG. 8). For example, as shown in FIG. 7, the inner wall 112 and outer wall 114 can be inclined towards one another as they move away from the reservoir 116, as represented by the extension lines 112a and 114a, respectively. The inner and outer walls 112, 114 can also have the same or similar angled configuration on the bottom side of the mouth piece 19. The angled configuration of the inner and outer walls 112, 114 can provide a comfortable snug fit of the mouth piece 19 with the user's gums while simultaneously providing a wider volume at the reservoir 116. The wider volume at the reservoir 116 can hold more whitening gel at the base of the teeth to provide more oxidizing agent, such as carbamide peroxide, to the teeth surfaces.

According to an illustrative embodiment, the mouth piece 19 may be sold as a pre-gelled unit with a high viscosity teeth whitening gel adhered to the top and bottom reservoirs 116, where the upper and lower rows of teeth will sit. Alternatively, the teeth whitening gel can be a lower viscosity type that the user can inject into the top and bottom reservoirs 116.

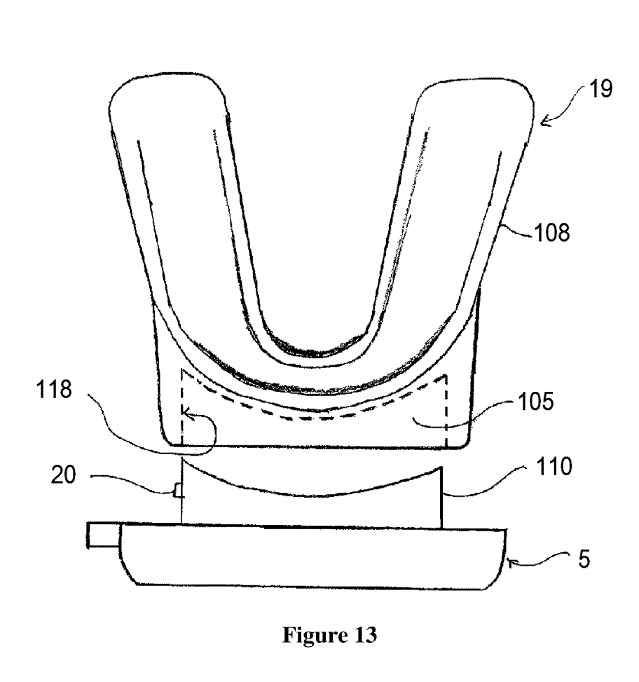

Referring to FIG. 10, the light housing 5 is shown coupled to the mouth piece 19. During use, the head unit 1 can be worn on the user's head, and the mouth piece 19 can be placed in the user's mouth. The light housing 5 can be moved on the extension member 4 (see FIG. 4) to line up with the mouth piece 19. The mouth piece 19 and the light housing 5 can be coupled together to transmit light from the light source (e.g., LEDs 104) through the mouth piece 19 and to the whitening gel and/or the user's teeth. For example, the plug portion 110 of the mouth piece 19 can slide into and engage the receptacle 105 of the light housing 5. Alternatively, as shown in FIG. 13, the plug portion 110 can be located on the light housing 5, and can engage a receptacle 105 located in the mouth piece 19. Either way, the plug portion 110 and the receptacle 105 can have substantially corresponding geometries. Once the light source is turned on, the plug portion 110 can act as a conduit that transmits light from the light source to the tray portion 108 of the mouth piece 19.

In the illustrative embodiment shown in FIGS. 7-10, the plug portion 110 comprises discreet first and second projections 110a and 110b that extend from the outer wall 114 of the mouth piece 19, however, other configurations are possible. For example, referring to the illustrative embodiment of FIG. 12, the plug portion 110 can comprise a single projection that extends across the front of the outer wall 114. Alternatively, the plug portion 110 can comprise more than two projections, or no projections at all. Still further, as shown in FIG. 13, the plug portion 110 can be located on the light housing 5, and the receptacle 105 can be located in the mouth piece 19.

The plug portion 110 and the receptacle 105 can have closely matching geometries such that the plug portion 110 slides into the receptacle 105 and snugly couples the two parts together. The plug portion 110 can transmit light from the light source to the tray portion 108 of the mouth piece 19. Other configurations to couple the light housing 5 and the mouth piece 10 will become apparent to those skilled in the art.

According to an illustrative embodiment of the system 100, when the mouth piece 19 is fully engaged with the light housing 5, six of the LEDs 104 may be located approximately 3.6 mm from the gel and teeth, and six of the LEDs 104 may be located approximately 6.8 mm from the gel and teeth, however, other configurations are possible. In addition, the reflective coating 109 may be provided on the interior surfaces of the receptacle 105, surrounding the LEDs 104. In this illustrative embodiment, the close proximity of the LEDs 104 to the teeth in conjunction with the reflective coating 109 guiding the light can produce a light power level on the teeth and whitening gel that would require a much more powerful light source when using conventional technologies in which the light source is positioned farther away from the patient. This higher power requirement may make other light sources impractical in a hands-free and/or portable device.

As an example, illustrative embodiments of the present invention can provide optical power levels to the teeth and whitening agent that are not reached by known portable devices. For example, according to an illustrative embodiment, the LEDs 104 can produce a minimum intensity of 11,000 millicandela (mcd) at a wavelength of approximately 470 nm and at a 50% power angle of approximately 15°. In this case, the total power produced by the LEDs in watts can be calculated using:

$$P = \frac{{I}_{c} π {tan}^{2} ( {θ}_{1 / 2} )}{( 683 ) V ( λ )}$$

where I_{c} is the intensity in mcd, θ_{1/2} is half the value of the 50% power angle, V(λ) is the photoptic luminous efficiency as a function of wavelength, and 683 lumens/watts is the conversion factor from lumens to watts.

The photoptic luminous efficiency can be found using a nonlinear curve fit to yield:

V(λ)=1.019e^{-285.4(λ-0.559)}^{²}

where λ is in microns.

Therefore, the illustrative embodiment utilizing a LED with an intensity of 11,000 mcd at a 50% power angle of 15° and a wavelength of 470 nm yields a total power for 12 LED's of 100 milliwatts.

According to an illustrative embodiment, insertion of the plug portion 110 into the receptacle 105 can activate the light source in the light housing 5. For example, as shown in FIGS. 4 and 11, a switch 20 can be located within the receptacle 105 in the light housing 5, for example, on an inner sidewall of shroud 106. The plug portion 110 of the mouth piece 19 can include a surface, such as the ramp surface 118 shown in FIGS. 7 and 12, that contacts the switch 20 when the plug portion 110 is substantially fully inserted into the receptacle 105.

Contact between the surface 118 and the switch 20 can cause the switch 20 (e.g., a biased switch) to move from an “off” position to an “on” position, thereby activating the light source. FIG. 13 shows an alternative embodiment where switch 20 is located on an outer surface of the light housing 5 (e.g., on the exterior of plug portion 110) and engages an interior surface of a receptacle 105 located in the mouth piece 19. The switch 20 can comprise any type of switch known in the art, such as, without limitation, a toggle switch, a pushbutton switch, a proximity switch, a pressure switch, or a temperature switch. Further, the switch 20 can comprise a plurality of switches that act in unison to activate and deactivate the light source. Removal of the plug portion 110 from the receptacle 105 can release the switch 20 to the “off” position, thereby deactivating the light source.

According to the illustrative embodiment with switch 20, the light source will not activate unless the mouth piece 19 is substantially fully inserted into receptacle 105 of the light housing 5. Likewise, the light source will deactivate whenever the mouth piece 19 is removed from the receptacle 105. This may help ensure that the light source is properly aligned with the teeth when in use, that the proper level of light intensity is impinging on the teeth, and/or that substantially no light will leave the light housing 5 without the mouth piece 19 in place, which may protect the customer from viewing unobstructed light from the light source. The switch 20 on the light housing 5 can be provided in addition to, or as an alternative to, the switch 102 on the power supply compartment 3.

As mentioned previously, the teeth whitening system 100 can include a teeth whitening agent, such as a whitening gel. The gel can absorb the light from the head unit 1 via light absorbing molecules in the gel called “activators.” The molecules absorb the light and are heated. This can result in a temperature rise in the whitening gel. The oxidation of hydrogen peroxide increases as a function of temperature. Therefore as the gel temperature increases, the bleaching action of the gel will be activated more rapidly, which can reduce the time needed to whiten the teeth.

According to an illustrative embodiment, the gel utilizes hydrogen peroxide as an oxidizing agent, such as carbamide peroxide mixed with an anhydrous viscous carrier containing glycerin and/or propylene glycol and/or polyethylene glycol. According to an illustrative embodiment, the strength of the carbamide peroxide is about 30-36%. When in contact with water, carbamide peroxide dissociates into urea and hydrogen peroxide. The hydrogen peroxide in the presence of water then dissociates into water and oxygen. It is the oxygen that reacts with the stains, making them more water soluble, transparent, or both. Other bleaching agents which can be used employ peroxyacetic acid and sodium perborate.

The activator may comprise, for example, any compound capable of absorbing light energy at biologically acceptable wavelengths. In general, such wavelengths are from about 350 nanometers (nm) to about 700 nm, encompassing a portion of the UVA spectrum (300 to 400 nm) and most of the visible light spectrum (400 to 700 nm). For safety, it may be favorable to have a gel activation wavelength in the visible part of the spectrum. According to an illustrative embodiment, the light can emit a biologically safe wavelength of visible light at about 470 nm, which can penetrate the teeth and react with activators in the whitening gel to speed the release of the oxidizing compound, thereby shortening the whitening process. According to an illustrative embodiment, the activator can comprise beta carotene, ferrous oxide, calcium chloride, or zinc oxide having a particle size between about 5 nm and about 20 nm.

The whitening gel can also include a thickening agent that imparts a medium to high viscosity to the formulation so that it can stay in the mouth piece 19. According to an illustrative embodiment, the thickening agent is inert and is not prone to oxidation by hydrogen peroxide.

The whitening gel can also include a flavorant. Flavorants are generally known in the art and can include, without limitation, spearmint, peppermint, citrus flavors, and vanilla. It may be desirable to provide within the compound an artificial sweetener such as sodium saccharin or potassium acesulfame.

According to an illustrative embodiment, the whitening gel can comprise a transparent solution or gel containing the oxidizing compound. This may allow certain wavelengths of light to be absorbed by tooth chromogens in a manner that promotes their oxidation to a non-chromogenic state. Therefore, according to an illustrative embodiment of the present invention, a tooth whitening method can include contacting the tooth enamel surface with an oxidizing compound, and thereafter exposing the tooth enamel surface to radiation corresponding to a tooth chromogen molecule absorption wavelength. Suitable wavelengths may include those between about 350 and about 700 nm, between about 380 and about 550 nm, and between about 400 and about 505 nm. According to an illustrative embodiment, an example wavelength for gel activators and chromogen molecular absorption may be about 470 nm.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A teeth whitening system, comprising:
a head unit adapted to be worn on a user's head;
a light housing supported by the head unit proximate to the user's mouth, the light housing including a light source; and
a mouth piece including a tray portion adapted to surround the user's teeth, wherein at least one of the light housing or the mouth piece includes a receptacle, and the other of the light housing or the mouth piece includes a plug portion adapted to engage in the receptacle.

2. The teeth whitening system of claim 1, further comprising:
a switch located on the light housing, wherein the switch is adapted to activate the light source upon engagement of the plug portion in the receptacle.

3. The teeth whitening system of claim 2, further comprising a plurality of switches adapted to act in unison to activate the light source upon engagement of the plug portion in the receptacle.

4. The teeth whitening system of claim 1, wherein the plug portion is located on the mouth piece, and the plug portion is adapted to transmit light from the light source to the tray portion of the mouth piece.

5. The teeth whitening system of claim 1, wherein the plug portion includes discreet first and second plug portions.

6. The teeth whitening system of claim 1, wherein the tray portion of the mouth piece includes an inner wall and an outer wall defining a gel reservoir, wherein the inner wall and the outer wall extend away from the gel reservoir and incline toward one another.

7. The teeth whitening system of claim 1, wherein the receptacle is located on the light housing, and includes a shroud that surrounds the light source to block light emitted by the light source from projecting outside the user's mouth.

8. The teeth whitening system of claim 1, further comprising a self-timer adapted to turn off the light source after a predetermined time period.

9. The teeth whitening system of claim 1, wherein the light source comprises an array of light emitting diodes located within the light housing.

10. The teeth whitening system of claim 1, further comprising a reflective coating surrounding the light emitting diodes.

11. The teeth whitening system of claim 1, further comprising a teeth whitening gel located in the tray portion of the mouth piece.

12. The teeth whitening system of claim 1, further comprising:
a power supply compartment supported on the head unit; and
an extension member coupling the light housing to the power supply compartment.

13. A teeth whitening apparatus, comprising:
a head unit adapted to be worn on a user's head;
a light housing supported by the head unit proximate to the user's mouth, the light housing including a light source, the light housing adapted to couple with a mouth piece; and
a switch located on the light housing, wherein the switch is adapted to activate the light source upon coupling of the light housing with the mouth piece.

14. The teeth whitening apparatus of claim 13, wherein the light housing includes a receptacle adapted to receive a portion of the mouth piece, and the switch is located inside the receptacle.

15. The teeth whitening apparatus of claim 14, wherein the receptacle includes a shroud that surrounds the light source to block light emitted by the light source from projecting outside the user's mouth, wherein the switch is located on an inner surface of the receptacle.

16. The teeth whitening apparatus of claim 14, further comprising a reflective coating located on an inner surface of the receptacle.

17. The teeth whitening apparatus of claim 13, further comprising a self-timer adapted to deactivate the light source after a predetermined time period.

18. The teeth whitening apparatus of claim 13, wherein the light source comprises an array of light emitting diodes.

19. The teeth whitening apparatus of claim 13, further comprising:
a power supply compartment supported on the head unit; and
an extension member coupling the light housing to the power supply compartment.

20. The teeth whitening apparatus of claim 13, further comprising a plurality of switches adapted to act in unison to activate the light source upon coupling of the light housing with the mouth piece.

21. A mouth piece for use in whitening teeth, comprising:
a tray portion including an inner wall and an outer wall adapted to surround a user's teeth; and
at least one of a plug portion or a receptacle extending from the tray portion to a position located outside the user's mouth, wherein at least one of the plug portion or the receptacle includes a surface adapted to engage a switch on a light housing to activate a light source.

22. The mouth piece of claim 21, wherein at least one of the plug portion or the receptacle is adapted to transmit light from the light source to the tray portion of the mouth piece.

23. The mouth piece of claim 21, wherein the plug portion includes discreet first and second plug portions.

24. The mouth piece of claim 21, wherein the tray portion includes an inner wall and an outer wall that define a gel reservoir, wherein the inner wall and the outer wall extend away from the gel reservoir and incline toward one another.

25. A method of whitening teeth, comprising:
inserting a mouth piece into a user's mouth;
placing a head unit on a user's head, the head unit supporting a light housing;
engaging the mouth piece with the light housing; and
channeling light from a light source located in the light housing to the user's teeth through the mouth piece.

26. The method of claim 25, wherein engaging the mouth piece with the light housing activates the light source.

27. The method of claim 26, wherein engaging the mouth piece with the light housing activates a switch located on the light housing to activate the light source.

28. The method of claim 26, wherein disengaging the mouth piece from the light housing deactivates the light source.

29. The method of claim 28, further comprising:
disengaging a switch on the light housing when disengaging the mouth piece from the light housing.

30. The method of claim 25, further comprising:
applying a teeth whitening gel to the mouth piece.
